(19) United States
(12) Patent Application Publication
(10) Pub. No.: US 2005/0150036 A1
(43) Pub. Date: Jul. 14, 2005

(54) Safety goggles assembly

(75) Inventor: Jimmy Chen, Taipei City (TW)

Correspondence Address: BRINKS HOFER GILSON & LIONE;NBC TOWER, Suite 3600, 455 N. Cityfront Plaza Drive, Chicago, IL, 60611-5599, US

(73) Assignee: Aswan International Corp.

(21) Appl. No.: 10/757,670

(22) Filed: Jan. 14, 2004

(51) Int. Cl. A61F 9/02

(52) U.S. Cl. 2/431

(57) ABSTRACT

A safety goggles assembly includes a protective lens having a peripheral edge portion formed with a plurality of through-holes, a support frame having a rim portion formed with a plurality of bifurcated parts, each of which defines opposite front and rear arms that sandwich the peripheral edge portion of the protective lens therebetween, and a plurality of connecting parts, each of which extends through a respective one of the through-holes in the protective lens to interconnect the front and rear arms of a respective one of the bifurcated parts, and an elastic strap interconnecting two opposite sides of a rear wall portion of the support frame.

### BACKGROUND OF THE INVENTION

1. Field of the Invention

This invention relates to a safety goggles assembly, more particularly to a safety goggles assembly having a support frame with a rim portion that is formed with bifurcated parts and connecting parts for retaining a protective lens in the rim portion.

2. Description of the Related Art

Conventional safety goggles assembly normally includes a protective lens and a support frame with a rim portion that defines a window for receiving the protective lens therein. The protective lens is secured to the rim portion in a press fit manner by inserting into a retaining recess in the rim portion, which is disadvantageous in that the protective lens tends to loosen after a period of use.

### SUMMARY OF THE INVENTION

Therefore, the object of the present invention is to provide a safety goggles assembly that is capable of overcoming the aforesaid drawback of the prior art.

According to the present invention, there is provided a safety goggles assembly that include: a protective lens having front and rear faces and a peripheral edge portion that is formed with a plurality of through-holes, each of which extends through the front and rear faces; a support frame having a rim portion and a rear wall portion that projects rearwardly from a periphery of the rim portion and that has two opposite sides, the rim portion defining a window for receiving the protective lens therein, and having a plurality of bifurcated parts, each of which defines opposite front and rear arms that are attached respectively to the front and rear faces of the protective lens so as to sandwich the peripheral edge portion of the protective lens therebetween, and a plurality of connecting parts, each of which extends through a respective one of the through-holes in the protective lens and each of which interconnects the front and rear arms of a respective one of the bifurcated parts; and an elastic strap interconnecting the sides of the rear wall portion of the support frame.

### BRIEF DESCRIPTION OF THE DRAWINGS

In drawings which illustrate an embodiment of the invention,

FIG. 1 is a perspective, partly cutaway view of the preferred embodiment of safety goggles assembly according to this invention;

FIG. 2 is a front view of a protective lens of the safety goggles assembly of FIG. 1;

FIG. 3 is a fragmentary sectional view showing engagement between the protective lens and a support frame of the safety goggles assembly of FIG. 1; and

FIG. 4 is a fragmentary sectional view to illustrate how a pair of holes are formed in the support frame during a molding process for forming the support frame on the protective lens of the safety goggles assembly of FIG. 1.

### DETAILED DESCRIPTION OF THE PREFERRED EMBODIMENT

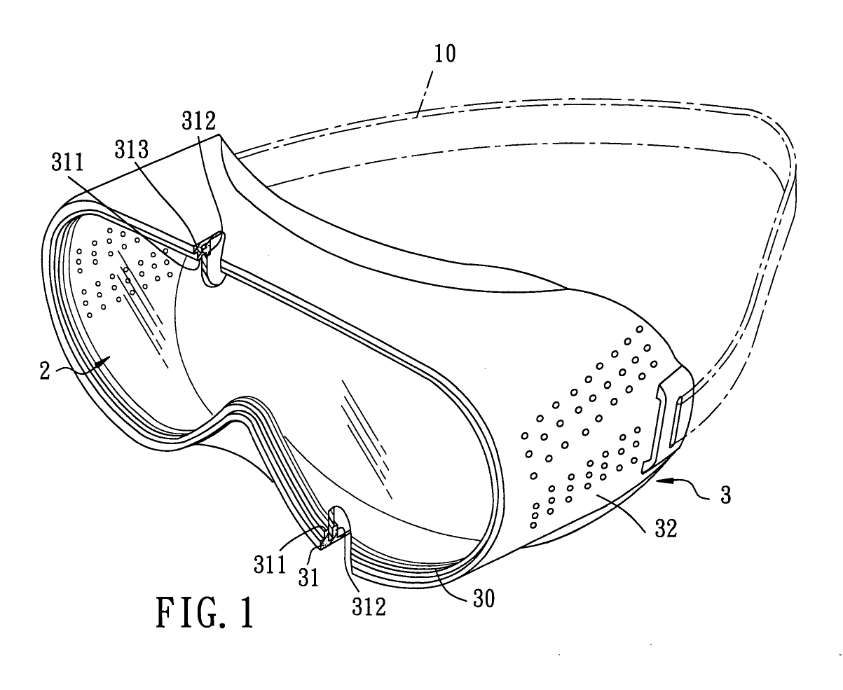

FIGS. 1 to 4 illustrate the preferred embodiment of a safety goggles assembly according to the present invention. The safety goggles assembly includes: a protective lens 2 having front and rear faces 25, 26 and a peripheral edge portion 24 that is formed with a plurality of through-holes 22, each of which extends through the front and rear faces 25, 26; a support frame 3 having a rim portion 31 and a rear wall portion 32 that projects rearwardly from a periphery of the rim portion 31 and that has two opposite sides, the rim portion 31 defining a window 30 for receiving the protective lens 2 therein, and having a plurality of bifurcated parts 33, each of which defines opposite front and rear arms 311, 312 that are attached respectively to the front and rear faces 25, 26 of the protective lens 2 so as to sandwich the peripheral edge portion 24 of the protective lens 2 therebetween, and a plurality of connecting part 313, each of which extends through a respective one of the through-holes 22 in the protective lens 2 and each of which interconnects and is integrally formed with the front and rear arms 311, 312 of a respective one of the bifurcated parts 33; and an elastic strap 10 interconnecting the sides of the rear wall portion 32 of the support frame 3.

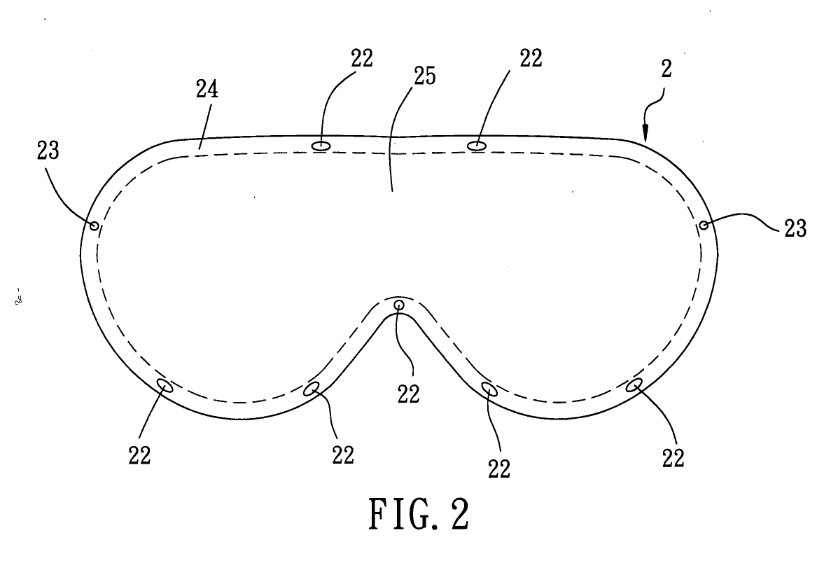

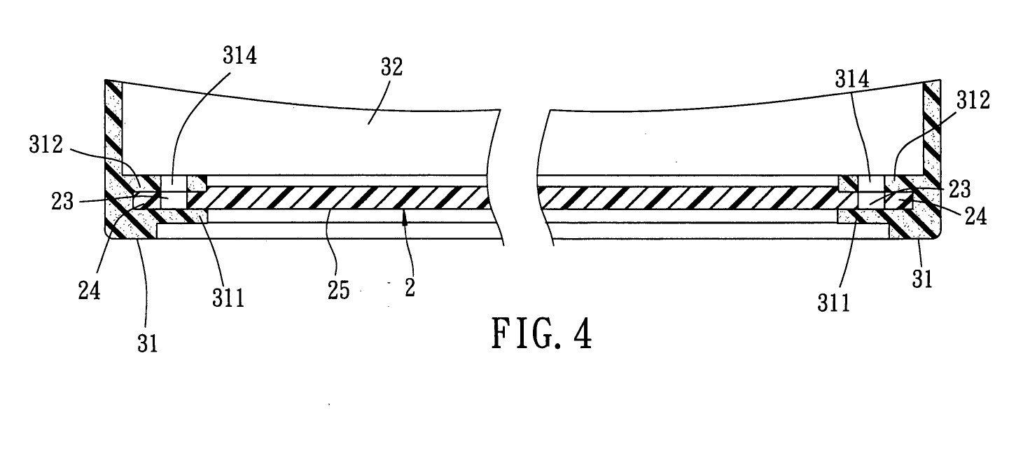

The support frame 3 is made from a moldable plastic material, and is continuously and integrally molded on the protective lens 2. Referring to FIGS. 2 and 4, a pair of positioning holes 23 are formed in the peripheral edge portion 24 of the protective lens 2 for positioning purposes during molding of the moldable plastic material on the protective lens 2 in a mold (not shown), which results in the formation of holes 314 in the rear arms 312 of corresponding bifurcated parts 33 in the support frame 3.

Preferably, the protective lens 2 is made from polycarbonate, and the moldable plastic material is polyvinyl chloride.

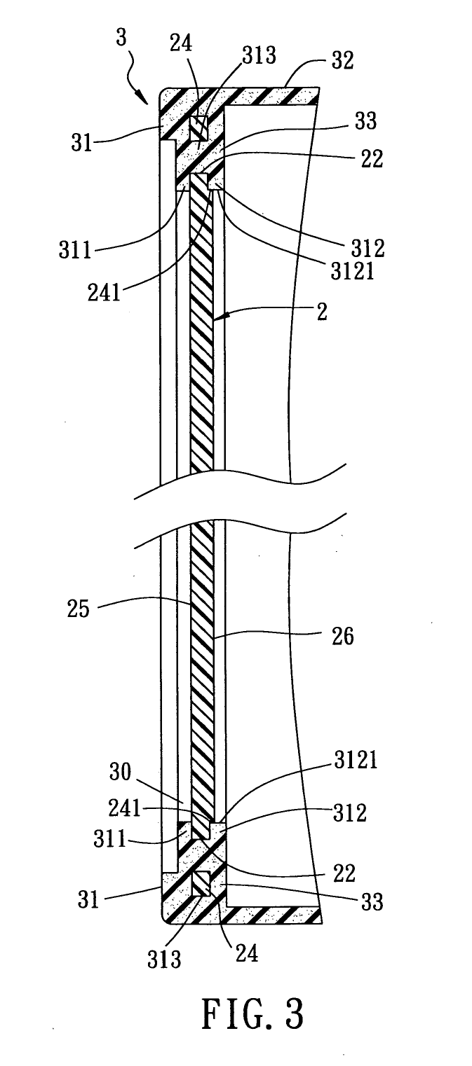

Referring back to FIG. 3, the rear arm 312 has an end face 3121 that faces toward the window 30. The peripheral edge portion 24 of the protective lens 2 is formed with a shoulder 241 that abuts against the end faces 3121 of the rear arms 312 of the bifurcated parts 33 of the rimportion 31 so as toprevent overflow of the moldable plastic material during the molding operation for forming the support frame 3.

Since the protective lens 2 can be firmly secured to the support frame 3 through the engagement with the connecting parts 313 and the bifurcated parts 33 and the engagement between the connecting parts 313 and the bifurcated parts 33, the aforesaid drawback as encountered in the prior art is thus eliminated.

With the invention thus explained, it is apparent that various modifications and variations can be made without departing from the spirit of the present invention.

1. Safety goggles assembly comprising:
a protective lens having front and rear faces and a peripheral edge portion that is formed with a plurality of through-holes, each of which extends through said front and rear faces; and
a support frame having a rim portion and a rear wall portion that projects rearwardly from a periphery of said rim portion and that has two opposite sides, said rim portion defining a window for receiving said protective lens therein, and having a plurality of bifurcated parts, each of which defines opposite front and rear arms that are attached respectively to said front and rear faces of said protective lens so as to sandwich said peripheral edge portion of said protective lens therebetween, and a plurality of connecting parts, each of which extends through a respective one of said through-holes in said protective lens and each of which interconnects said front and rear arms of a respective one of said bifurcated parts.

2. The safety goggles assembly of claim 1, further comprising an elastic strap interconnecting said sides of said rear wall portion of said support frame.

3. The safety goggles assembly of claim 1, wherein said support frame is made from a moldable plastic material, and is continuously and integrally molded on said protective lens.

4. The safety goggles assembly of claim 3, wherein said protective lens is made from polycarbonate, said moldable plastic material is polyvinyl chloride.

5. The safety goggles assembly of claim 1, wherein said rear arm has an end face that faces toward said window, said peripheral edge portion of said protective lens being formed with a shoulder that abuts against said end faces of said rear arms of said bifurcated parts of said rim portion.
